(19) United States
(12) Patent Application Publication
(10) Pub. No.: US 2009/0275971 A1
(43) Pub. Date: Nov. 5, 2009

(54) Energy activated preloaded detachment mechanisms for implantable devices

(75) Inventors: Brian Kelleher, Del Mar, CA (US); Matt Yurek, San Diego, CA (US); Corbett Stone, San Diego, CA (US)

Correspondence Address: ROBINS & PASTERNAK, 1731 EMBARCADERO ROAD, SUITE 230, PALO ALTO, CA, 94303, US

(73) Assignee: Boston Scientific Scimed, Inc.

(21) Appl. No.: 12/290,651

(22) Filed: Oct. 30, 2008

Related U.S. Application Data
(60) Provisional application No. 61/000,971, filed on Oct. 30, 2007.

(51) Int. Cl. A61M 29/00

(52) U.S. Cl. 606/191

(57) ABSTRACT

Described herein are energy-activated preloaded detachment mechanisms for implantable devices and assemblies comprising these detachment mechanisms. Also provided are methods of using the detachment mechanisms and assemblies.

### CROSS-REFERENCE TO RELATED APPLICATIONS

This application claims the benefit of U.S. provisional patent application No. 61/000,971, filed Oct. 30, 2007, the disclosure of which is incorporated by reference in its entirety for all purposes.

### TECHNICAL FIELD

This disclosure relates to degradable detachment mechanisms for implantable devices.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings that may be dimensioned to engage the walls of the vessels. (See, e.g., U.S. Pat. No. 4,994,069 to Ritchart et al.). Variations of such devices include polymeric coatings or attached polymeric filaments have also been described. See, e.g., U.S. Pat. Nos. 5,226,911; 5,935,145; 6,033,423; 6,280,457; 6,287,318; and 6,299,627. In addition, coil designs including stretch-resistant members that run through the lumen of the helical vaso-occlusive coil have also been described. See, e.g., U.S. Pat. Nos. 5,582,619; 5,833,705; 5,853,418; 6,004,338; 6,013,084; 6,179,857; and 6,193,728.

Typically, implantable devices include a detachment mechanism in order to be released from the deployment mechanism (e.g., attached wire). Several classes of techniques have been developed to enable more accurate placement of implantable devices within a vessel. One class involves the use of electrolytic means to detach the vasoocclusive member from the pusher. Electrolytic coil detachment is disclosed in U.S. Pat. Nos. 5,122,136; 5,354,295; 6,620,152; 6,425,893; and 5,976,131, all to Guglielmi et al., describe electrolytically detachable embolic devices. U.S. Pat. No. 6,623,493 describes vaso-occlusive member assembly with multiple detaching points. U.S. Pat. Nos. 6,589,236 and 6,409,721 describe assemblies containing an electrolytically severable joint. The coil is bonded via a metal-to-metal joint to the distal end of the pusher. The pusher and coil are made of dissimilar metals. The coil-carrying pusher is advanced through the catheter to the site and a small electrical current is passed through the pusher-coil assembly. The current causes the joint between the pusher and the coil to be severed via electrolysis. The pusher may then be retracted leaving the detached coil at an exact position within the vessel. Since no significant mechanical force is applied to the coil during electrolytic detachment, highly accurate coil placement is readily achieved. In addition, the electric current may facilitate thrombus formation at the coil site. The disadvantage of this method is that the electrolytic release of the coil may require a period of time that may inhibit rapid detachment of the coil from the pusher.

Other forms of energy are also used to sever sacrificial joints that connect pusher and vasoocclusive member apparatus. Sacrificial connection member, preferably made from polyvinylacetate (PVA), resins, or shape memory alloys, can be used to join a conductive wire to a detention member. See, U.S. Pat. Nos. 5,759,161 and 5,846,210. Upon heating by a monopolar high frequency current, the sacrificial connection member melts, severing the wire from the detention member.

U.S. Pat. No. 5,944,733 describes application of radiofrequency energy to sever a thermoplastic joint and U.S. Pat. No. 6,743,251 describes detachment joints that are severed by the application of low frequency energy or direct current. U.S. Pat. No. 6,346,091 describes a wire detachment junction that is severed by application of vibrational energy.

In U.S. Pat. No. 4,735,201 to O'Reilly, an optical fiber is enclosed within a catheter and connected to a metallic tip on its distal end by a layer of hot-melt adhesive. The proximal end of the optical fiber is connected to a laser energy source. When endovascularly introduced into an aneurysm, laser energy is applied to the optical fiber, heating the metallic tip so as to cauterize the immediately surrounding tissue. The layer of hot-melt adhesive serving as the bonding material for the optical fiber and metallic tip is melted during this lasing, but the integrity of the interface is maintained by application of back pressure on the catheter by the physician. When it is apparent that the proper therapeutic effect has been accomplished, another pulse of laser energy is then applied to once again melt the hot-melt adhesive, but upon this reheating the optical fiber and catheter are withdrawn by the physician, leaving the metallic tip in the aneurysm as a permanent plug.

Other methods for placing implantable devices within the vasculature utilize heat releasable bonds that can be detached by using laser energy (see, U.S. Pat. No. 5,108,407). EP 0 992 220 describes an embolic coil placement system which includes conductive wires running through the delivery member. When these wires generate sufficient heat, they are able to sever the link between the embolic coil and the delivery wires. Further, U.S. Pat. No. 6,113,622 describes the use of fluid pressure (e.g., hydraulics) to detach an embolic coil.

A variety of mechanically detachable devices are also known. For instance, U.S. Pat. No. 5,234,437, to Sepetka, shows a method of unscrewing a helically wound coil from a pusher having interlocking surfaces. U.S. Pat. No. 5,250,071, to Palermo, shows an embolic coil assembly using interlocking clasps mounted both on the pusher and on the embolic coil. U.S. Pat. No. 5,261,916, to Engelson, shows a detachable pusher-vaso-occlusive coil assembly having an interlocking ball and keyway-type coupling. U.S. Pat. No. 5,304,195, to Twyford et al., shows a pusher-vaso-occlusive coil assembly having an affixed, proximally extending wire carrying a ball on its proximal end and a pusher having a similar end. The two ends are interlocked and disengage when expelled from the distal tip of the catheter. U.S. Pat. No. 5,312,415, to Palermo, also shows a method for discharging numerous coils from a single pusher by use of a guidewire which has a section capable of interconnecting with the interior of the helically wound coil. U.S. Pat. No. 5,350,397, to Palermo et al., shows a pusher having a throat at its distal end and a pusher through its axis. The pusher sheath will hold onto the end of an embolic coil and will then be released upon pushing the axially placed pusher wire against the member found on the proximal end of the vaso-occlusive coil.

However, there remains need for alternative detachment mechanisms, particularly energy-activated preloaded detachment mechanisms.

### SUMMARY

Described herein are energy-activated preloaded detachment mechanisms. In particular, the detachment mechanisms include a preloaded source of potential energy (e.g., spring, fluid, vacuum, etc.) which is held in a first locked (preloaded) position by an energy-activated locking mechanism. In the first (locked) position, the detachment mechanism secures an implantable device within a delivery device. When the locking mechanism is activated by the application of energy, the preloaded source of energy switches to the second (unlocked) position. This change in configuration releases the potential energy from the preloaded source of energy and release of this energy directly or indirectly effects deployment of the implantable device.

In certain aspects, disclosed herein is an assembly for an implantable device comprising: a delivery device; an implantable device; an element having first and second positions, wherein, in the first position, the element (i) holds the implantable device within the delivery device and (ii) comprises a source of potential energy; and an energy-activated locking mechanism for changing the element from the first and second positions upon the application of energy. The assembly may further comprise a sleeve surrounding at least a portion of the implantable device and, in certain embodiments, may further comprise a jaw structure within the sleeve, wherein the jaw structure secures the implantable device when the element having first and second positions is in the first position.

In any of the assemblies described herein, the energy-activated locking mechanism may be, for example, salt, sugar, glass, one or more polymers (e.g., poly-L-lactic acid (PLLA), polyglycolic acid (PGA), polyvinyl alcohol (PVA) and/or combinations thereof), lipids, crystal structures, tetrahedrons, and combinations thereof.

In certain embodiments, the element having first and second positions comprises a spring (e.g., a compression spring, an extension spring and/or a twisted spring). In other embodiments, the element having first and second positions comprises a pressurized fluid. In still other embodiments, the element having first and second positions comprises a vacuum.

Any of the assemblies described herein may further comprise means for applying energy to activate the locking mechanism, for example, a source of electromagnetic radiation (e.g., radio waves, microwaves, terahertz radiation, infrared radiation, visible light, ultraviolet radiation, X-rays, gamma rays and combinations thereof), thermal energy, electrical energy, vibrational energy (e.g., ultrasonic), and combinations thereof.

In any of the assemblies described herein, the implantable device may comprise a vaso-occlusive device, for example a vaso-occlusive coil or a tubular braid. Furthermore, any of the assemblies described herein may further comprise a delivery device (e.g., catheter, microcatheter, etc.).

In another aspect, described herein is a method of occluding a body cavity, the method comprising introducing one or more of any of the implantable assemblies described herein into the body cavity. In certain embodiments, the body cavity is an aneurysm.

These and other embodiments will readily occur to those of skill in the art in light of the disclosure herein.

### BRIEF DESCRIPTION OF THE FIGURES

FIG. 1 is a side view of an exemplary assembly as described herein. The preloaded source of potential energy comprises a spring. FIG. 1 shows the spring in a first (extended) position. The embodiment shown in FIG. 1 includes a hinged jaw structure within a sleeve that holds the implantable device in place when the spring is in a first (locked) position.

FIG. 2 is a side view of the exemplary assembly of FIG. 1 shown when the spring releases its potential energy as it compresses and the released energy moves a sleeve holding the implantable device such that the hinged jaws open and the implant is deployed.

FIG. 3 is a side-view of another exemplary assembly as described herein. In the embodiment shown, the implantable device directly contacts and is held in place by a sleeve prior to activation of the locking mechanism with energy.

FIG. 4 is a side-view of the exemplary assembly of FIG. 3 shown when the spring is allowed to compress, it releases its potential energy and moves the sleeve holding that implantable device such that the implant is deployed from the delivery device.

FIG. 5 is a side-view of another exemplary assembly as described herein in which the sleeve holding the implantable device is secured to the implant.

FIG. 6 is a side-view of the exemplary assembly of FIG. 5 after unlocking of the locking mechanism by application of energy. The spring compresses and the implant and attached sleeve are both released into the selected site.

FIG. 7 is a side-view of yet another exemplary assembly in which the preloaded source of energy comprises a pressurized fluid or vacuum.

FIG. 8 is a side view of the exemplary assembly of FIG. 7 shown when the pressurized fluid or vacuum is released and, as it compresses, moves a sleeve holding the implantable device such that the implant is deployed.

### DETAILED DESCRIPTION

Detachment mechanisms for implantable devices and assemblies comprising these detachment mechanisms are described. The detachment mechanisms described herein find use in deploying vascular and neurovascular implants and are particularly useful in treating aneurysms, for example small-diameter, curved or otherwise difficult to access vasculature, for example aneurysms, such as cerebral aneurysms. Methods of making and using these detachment mechanisms and assemblies are also described.

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise.

The detachment mechanisms described herein allow for rapid and precise detachment of an implantable device upon application of energy by triggering a cascade resulting in deployment of the implantable device. In particular, the detachment mechanisms are configured so that an energy-activated locking mechanism holds a source of potential energy in a position where the potential energy is stored. When the operator activates (unlocks) the locking mechanism by applying energy, the potential energy stored in the preloaded source of potential energy is released, for example by a change in configuration of this element. The potential energy released by the preloaded source of energy in turn causes release of the implantable device.

Release of the implant can be accomplished in any number of ways. In certain embodiments, the release of potential energy caused by unlocking the locking mechanism causes a sleeve surrounding the implant to be withdrawn, allowing deployment of the implant. The sleeve may directly contact the interior (lumen) or, as shown in the Figures, the exterior of the implant. Alternatively, the sleeve may contact another element (e.g., jaws) that secure the implant in the delivery device when the preloaded source of energy is in the first (potential energy containing) state. Furthermore, it will be apparent that configurations other than sleeves can be employed, for example designs in which the potential energy released when the energy-activated locking mechanism is unlocked pushes, pulls and/or rotates the implant directly or indirectly via an element (e.g., sleeve) that contacts the implant.

Any preloaded source of potential energy can be used can be used in the detachment mechanisms described herein, including, but not limited to, one or more springs (extension, twisted or compression), pressurized fluid, a vacuum, or the like. For example, the preloaded source of energy can comprise a spring held in an extended preloaded position by a locking mechanism. Upon activation of the locking mechanism, the spring contracts and releases its potential energy. Alternatively, the preloaded source of energy can comprise a pressurized fluid or a vacuum that is released upon activation (e.g., melting) of a locking mechanism holding the fluid or vacuum.

Similarly, any energy-activated locking mechanism can be employed in the devices and assemblies described herein. By “energy-activated” in reference to the locking mechanism is meant any material in a configuration holds the source of potential energy in place prior to application of energy and, upon application of energy is sufficiently degraded, dissolved, melted, fluidized, or the like to unlock and release the source of potential energy. Non-limiting examples of suitable energy activated materials include naturally occurring materials, synthetic materials or combinations of natural and synthetic materials, such as salt, sugar, glass, polymers (e.g., poly-L-lactic acid (PLLA), polyglycolic acid (PGA), polyvinyl alcohol (PVA), as well as other energy activated polymers known to those of skill in the art), lipids (e.g., cholesterol), other crystal structures and/or tetrahedron materials. In any of the embodiments described herein, the locking mechanism may be secured to the element having a source of potential energy and/or to an optional sleeve. Alternatively, the locking mechanism may be secured to the implantable device, for example to a sleeve that is secured to the device.

Examples of suitable forms of energy for unlocking the locking mechanism and releasing the potential energy include, but are not limited to, electromagnetic radiation (e.g., radio waves, microwaves, terahertz radiation, infrared radiation, visible light, ultraviolet radiation, X-rays and gamma rays), heat (thermal) energy, electrical energy, vibrational energy (e.g., sonic or ultrasonic) and combinations thereof.

Delivery mechanisms (e.g., catheter or delivery tube) that allow for energy to be transmitted to the locking mechanism include, for example, multi-lumen catheters for transmitting fluids and catheters comprising energy conductors (e.g., electrodes or heat conductors) in the side-walls. See, e.g., U.S. Pat. Nos. 6,059,779 and 7,020,516. Conductors of the degradation substance may also be transmitted through the lumen of the delivery mechanism. For example, bi-polar electrodes and/or anodes alone or twisted with a core wire cathode can also be used to supply current to the locking element. The conductive element may include a polymer jacket/liner to insulate the conductors and/or reduce friction during advancement. Thus, the energy or other substances that induce degradation can be from the proximal end of the delivery device to the energy-activated locking mechanism via such conductors.

Depicted in the appended drawings are exemplary embodiments of the present invention in which the implantable device is depicted as an embolic device. It will be appreciated that the drawings are for purposes of illustration only and that other implantable devices can be used in place of embolic devices, for example, stents, filters, and the like. Furthermore, although depicted in the Figures as embolic coils, the embolic devices may be of a variety of shapes or configuration including, but not limited to, braids, wires, knits, woven structures, tubes (e.g., perforated or slotted tubes), injection-molded devices and the like. See, e.g., U.S. Pat. No. 6,533,801 and International Patent Publication WO 02/096273. It will also be appreciated that the assemblies can have various configurations as long as the required flexibility is present.

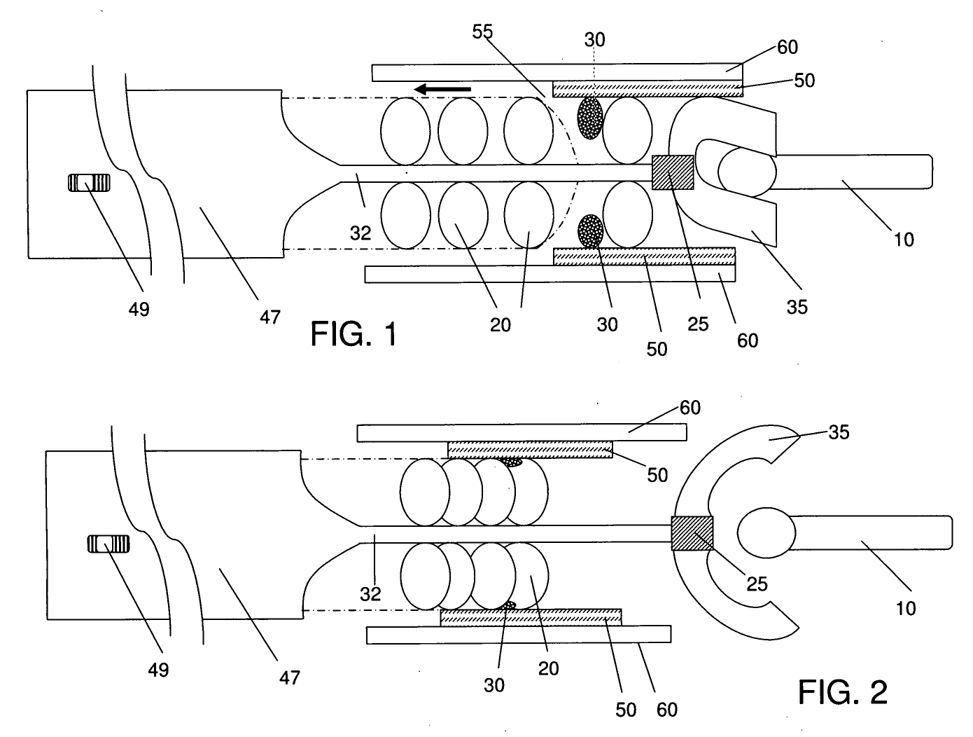

FIG. 1 is a side and view of an exemplary assembly as described herein. The implantable coil 10 is held in place by hinged 25 jaws 35 within a sleeve 50 by a locking mechanism 30 secured to the sleeve 50. Prior to activating locking mechanism 30, sleeve 50 holds jaws 35 closed around implant 10 because locking mechanism 30 keeps spring 20 in its extended position. Also shown are deployment device 60 (e.g., catheter, delivery tube), heat bore 55, energy conducting element 32 for activating locking mechanism 30, energy source 47, and actuator 49 for application of energy by the operator. The arrow shows the direction of movement of the spring 20 when it is released.

Energy conducting element 32 will be any configuration and material that allows for delivery of the activating energy. For example, the conductor element may comprise a conductive material such as stainless steel, platinum, gold, etc. One or more conductor elements may be present. Furthermore, although shown in the Figures as positioned in the lumen of the delivery device, it will be apparent that the conductor element 32 can be positioned in the sidewalls of the selected delivery device 60.

FIG. 2 shows the exemplary assembly of FIG. 1 after the locking mechanism 30 is activated by application of energy. When the spring 20 is no longer held in the preloaded position, it compresses toward the distal end of the assembly and also brings the activated locking mechanism 30 and attached sleeve 50 distally. When the sleeve 50 no longer holds the hinged 25 jaws 35 closed, the jaws 35 open and the implantable device 10 is deployed.

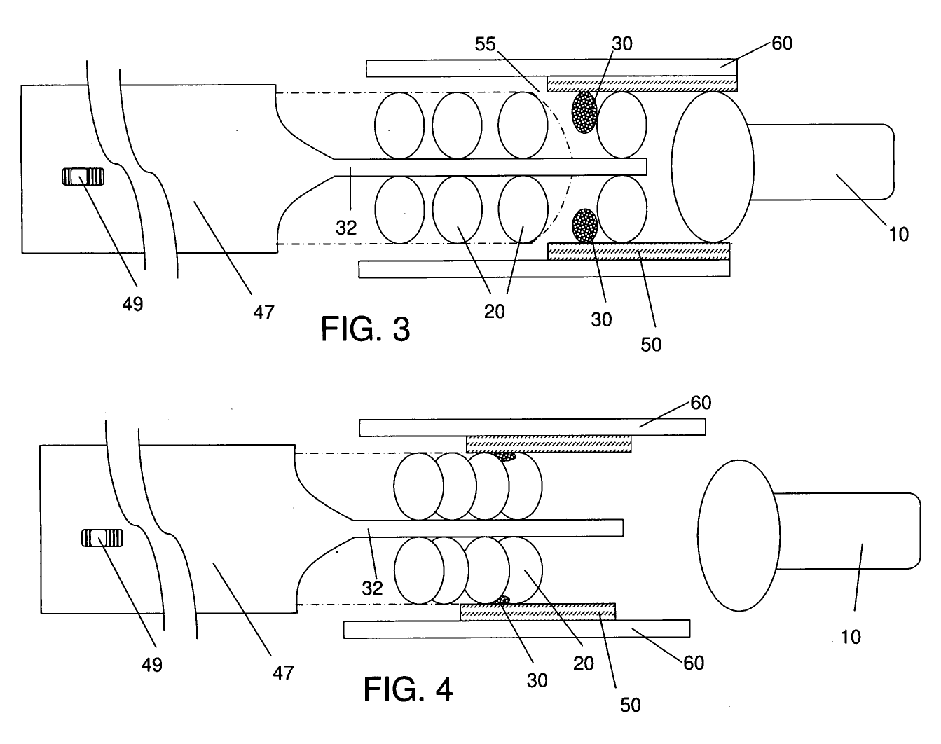

FIG. 3 shows another exemplary embodiment in the locked position. The implantable device 10 is held within delivery device 60 directly by sleeve 50 by locking mechanism 30 in the locked position. The locking mechanism 30 may be secured to sleeve 50 and/or to spring 20.

FIG. 4 shows the assembly of FIG. 3 after application of energy to unlock the locking mechanism 30. The spring 20 and sleeve 50 move distally, releasing the implant 10.

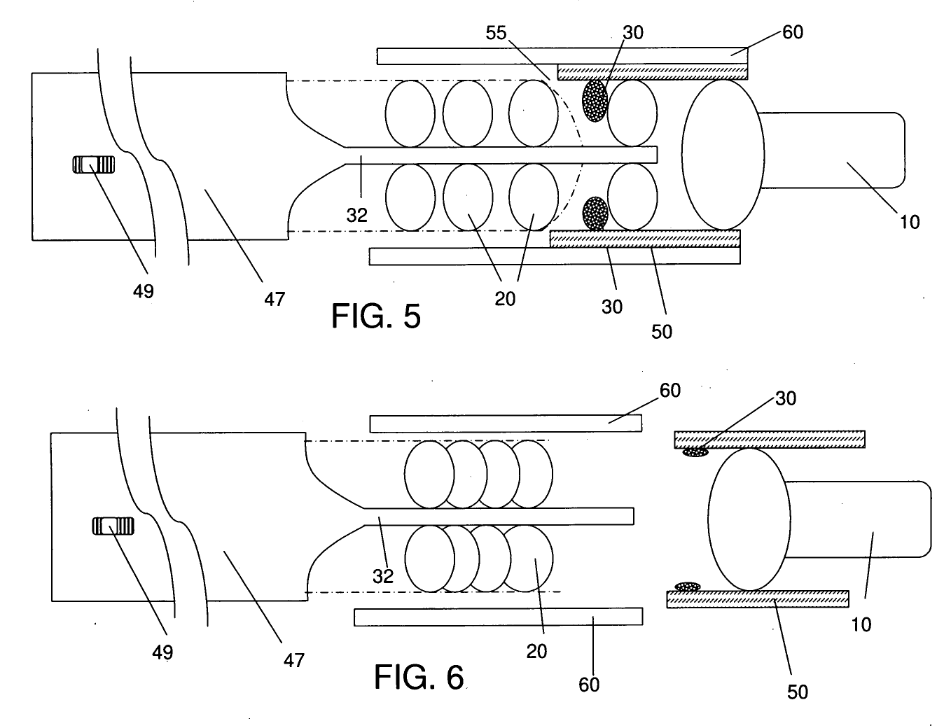

FIG. 5 shows an embodiment in which the energy-activated locking mechanism 30 is secured to implant 10 via sleeve 50. The assembly is shown in the locked position in which the spring 20 is extended.

As shown in FIG. 6, upon application of energy, locking mechanism 30 releases potential energy of spring 20 as it compresses and the implant 10 and attached sleeve 50 are deployed.

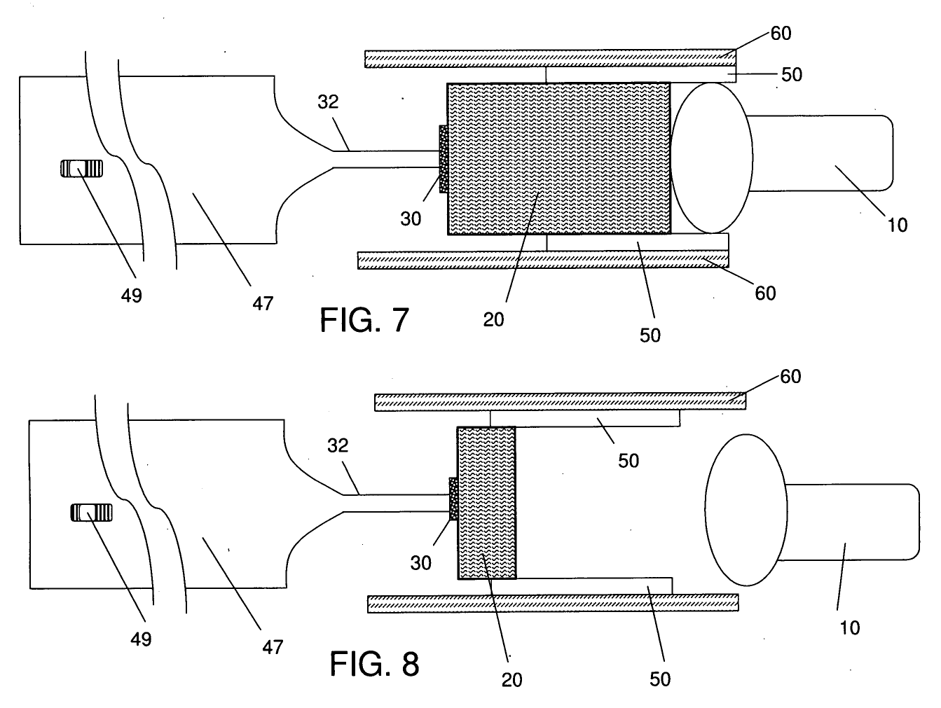

FIG. 7 is a side and view of an exemplary assembly as described herein. The implantable coil 10 is held in place by sleeve 50 secured to a reservoir 20 of pressurized fluid or a vacuum. Prior to activation with energy, the locking mechanism 30 maintains the pressure within the reservoir. Also shown are deployment device 60 (e.g., catheter, delivery tube), energy conducting element 32 for activating locking mechanism 30, energy source 47, and actuator 49 for application of energy by the operator.

FIG. 8 shows the exemplary assembly of FIG. 7 after the locking mechanism 30 is activated (melted, dissolved, fluidized, etc.). The activation of the locking mechanism 30 releases the pressure (e.g., by releasing the fluid into the delivery device) from the reservoir 20, which compresses toward the distal end of the assembly, bringing the sleeve 50 distally. When the sleeve 50 no longer holds the implantable device 10, the implant is deployed.

With regard to particular materials used in the implantable devices and assemblies of the invention, it is to be understood that the implantable devices or assemblies may be made of a variety of materials, including but not limited to metals, polymers and combinations thereof, including but not limited to, stainless steel, platinum, kevlar, PET, carbothane, cyanoacrylate, epoxy, poly(ethyleneterephthalate) (PET), polytetrafluoroethylene (Teflon™), polypropylene, polyimide polyethylene, polyglycolic acid, polylactic acid, nylon, polyester, fluoropolymer, and copolymers or combinations thereof. See, e.g., U.S. Pat. Nos. 6,585,754 and 6,280,457 for a description of various polymers. Different components of the devices and assemblies may be made of different materials.

In embodiments in which the implantable device comprises an embolic coil, the main coil may be a coiled and/or braided structure comprising one or more metals or metal alloys, for example, Platinum Group metals, especially platinum, rhodium, palladium, rhenium, as well as tungsten, gold, silver, tantalum, stainless steel and alloys of these metals. Preferably, the comprises a material that maintains its shape despite being subjected to high stress, for example, “super-elastic alloys” such as nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.” The main coil may also comprise a shape memory polymer such as those described in International Publication WO 03/51444. The implantable device is preferably electrically insulated, for example, by coating a metallic coil (e.g., stainless steel, platinum) with one or more electrically insulating materials, for example one or more polymers such as polyimide.

The implantable device may also change shape upon release from the deployment mechanism (e.g., pusher wire), for example change from a linear form to a relaxed, three-dimensional configuration upon deployment.

The devices described herein may also comprise additional components, such as co-solvents, plasticizers, coalescing solvents, bioactive agents, antimicrobial agents, antithrombogenic agents (e.g., heparin), antibiotics, pigments, radiopacifiers and/or ion conductors which may be coated using any suitable method or may be incorporated into the element(s) during production. See, e.g., U.S. Pat. No. 6,585,754 and WO 02/051460, U.S. Pat. No. 6,280,457. The additional components can be coated onto the device and/or can be placed in the vessel prior to, concurrently or after placement of one or more devices as described herein.

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared and/or flushed with an electrolyte solution.

Once the selected site has been reached, the vaso-occlusive device is extruded using a pusher-detachment mechanism as described herein and released in the desired position of the selected site.

Modifications of the procedures and assemblies described above, and the methods of using them in keeping with this disclosure will be apparent to those having skill in this mechanical and surgical art. These variations are intended to be within the scope of the claims that follow.

What is claimed is:

1. An assembly for an implantable device comprising:
a delivery device;
an implantable device;
an element having first and second positions, wherein, in the first position, the element (i) holds the implantable device within the delivery device and (ii) comprises a source of potential energy; and
an energy-activated locking mechanism for changing the element from the first and second positions upon the application of energy.

2. The assembly of claim 1, further comprising a sleeve surrounding at least a portion of the implantable device.

3. The assembly of claim 2, further comprising a jaw structure within the sleeve, wherein the jaw structure secures the implantable device when the element having first and second positions is in the first position.

4. The assembly of claim 1, wherein the energy-activated locking mechanism is selected from the group consisting of salt, sugar, glass, one or more polymers, lipids, crystal structures, tetrahedrons, and combinations thereof.

5. The assembly of claim 3, wherein the energy-activated locking mechanism comprises a polymer selected from the group consisting of poly-L-lactic acid (PLLA), polyglycolic acid (PGA), polyvinyl alcohol (PVA) and combinations thereof.

6. The assembly of claim 1, wherein the element having first and second positions comprises a spring.

7. The assembly of claim 6, wherein the spring is selected from the group consisting of a compression spring, an extension spring and a twisted spring.

8. The assembly of claim 1, wherein the element having first and second positions comprises a pressurized fluid.

9. The assembly of claim 1, wherein the element having first and second positions comprises a vacuum.

10. The assembly of claim 1, further comprises means for applying energy to activate the locking mechanism.

11. The assembly of claim 10, wherein the means for applying energy comprises a conductor of energy selected from the group consisting of electromagnetic radiation, thermal energy, electrical energy, vibrational energy, and combinations thereof.

12. The assembly of claim 11, wherein the energy is electromagnetic radiation and the electromagnetic radiation is selected from the group consisting of radio waves, microwaves, terahertz radiation, infrared radiation, visible light, ultraviolet radiation, X-rays, gamma rays and combinations thereof.

13. The assembly of claim 11, wherein the energy is thermal energy.

14. The assembly of claim 11, wherein the energy is vibrational energy.

15. The assembly of claim 1, wherein the implantable device comprises a vaso-occlusive device.

16. The assembly of claim 15, wherein the vaso-occlusive device is a coil or a tubular braid.

17. The assembly of claim 1, wherein the delivery device comprises a catheter.

18. The assembly of claim 17, wherein the delivery device comprises a hypotube.

19. A method of occluding a body cavity comprising introducing an implantable assembly according to claim 1 into the body cavity.

20. The method of claim 19, wherein the body cavity is an aneurysm.
